(19) United States
(12) Patent Application Publication
(10) Pub. No.: US 2014/0276522 A1
(43) Pub. Date: Sep. 18, 2014

(54) PROTECTIVE DISPOSABLE WRAPPER FOR TAMPONS

(71) Applicant: Carrie Thompson, Portland, OR (US)

(72) Inventor: Carrie Thompson, Portland, OR (US)

(21) Appl. No.: 14/214,059

(22) Filed: Mar. 14, 2014

Related U.S. Application Data
(60) Provisional application No. 61/799,357, filed on Mar. 15, 2013.

(51) Int. Cl. A61F 13/551

(52) U.S. Cl. CPC A61F 13/5518
USPC 604/385.02

(57) ABSTRACT

A tampon enclosure system comprising a pocket which encloses all or part of a user's hand during removal and encloses a used tampon during disposal.

### CROSS REFERENCE TO RELATED APPLICATIONS

The present application claims priority to U.S. Provisional Patent Application No. 61/799,357, filed Mar. 15, 2013, entitled “PROTECTIVE DISPOSABLE WRAPPERS FOR TAMPONS,” the entire disclosure of which is hereby incorporated by reference in its entirety.

### BACKGROUND OF THE INVENTION

Tampons were invented in 1929, and have been used regularly by millions of women for decades. While advances in applicators have made insertion of tampons relatively easy, clean, and comfortable, removal and disposal of used tampons remain very unsanitary.

Disposal is problematic. Flushing tampons down a toilet clogs plumbing, and discarding a used tampon in a trash can is unsanitary, as it introduces blood and other bodily fluids into the environment where they can be a source of contamination. Women often wrap used tampons in toilet paper or paper towels to make trash can disposal somewhat less unsanitary, but as both of those materials are porous, this practice accomplishes little besides wasting toilet paper. Used tampons are the only blood-contaminated biohazards customarily disposed of in open containers in public spaces.

Removal is also problematic. Users must find the string used for extraction which is often entangled in labia and can be challenging to identify and extricate. This process can be very unsanitary. Many women use toilet paper as a barrier for their fingers, but toilet paper is not an effective barrier and can interfere with a user's ability to find the string. A user's fingers can become soiled during extraction. If a user is in a public restroom stall, she must fasten her clothing before she can wash her hands, and she risks staining her clothes, which in addition to harming her clothing can be extremely embarrassing. Microbes or contaminants present on a user's fingers during extraction can cause vaginal infections or labial irritation. When camping or using a portable outdoor toilet, hand washing is not possible.

Additionally, current packaging for conventional individual tampons is problematic. Many women place tampons in their purses for later use, where they remain for some period of time. They get damaged by friction with other objects in the purse, and when they are eventually needed, days or weeks after being initially placed in the purse, they are dirty and damaged and often unusable.

All of these problems have been well known to users of tampons for decades, but despite this long felt need, no commercial product has been developed which adequately addresses these issues.

### BRIEF DESCRIPTION OF THE INVENTION

Disclosed herein is a tampon enclosure system comprising: a tampon; a first pocket and a second pocket, said first pocket being removably joined to the second pocket; said first pocket being sized and shaped to accommodate said tampon; and said second pocket being sized and shaped to accommodate a hand of a tampon user.

The foregoing and other objectives, features, and advantages of the invention will be more readily understood upon consideration of the following detailed description of the invention taken in conjunction with the accompanying drawings.

### BRIEF DESCRIPTION OF THE DRAWINGS

FIG. 1A is a perspective view of a tampon enclosed in one embodiment of the enclosure system described herein and in a secondary wrapper;

FIG. 1B is a perspective view of a secondary wrapper with the one end torn open and the tampon, encased in enclosure system, removed.

FIG. 2 is a plan view of a tampon partially enclosed in the first pocket of one embodiment of the tampon enclosure system disclosed herein.

FIG. 3 is a plan view of the first pocket of one embodiment of a tampon enclosure system, with tampon enclosed, being separated from the second pocket along a perforated seam.

FIG. 4 is a plan view of the first pocket as shown in FIG. 3, separated from the first pocket, open at one end so that the tampon may be removed.

FIG. 5 is a plan view of a user's hand inserted within a second pocket of one embodiment of the tampon enclosure system described herein.

FIG. 6A is a perspective view showing a user grasping the string of a tampon, and withdrawing the tampon;

FIG. 6B is a perspective view showing a user preparing to pull the second pocket over the tampon.

FIG. 7 is a perspective view showing the second pocket pulled over the tampon;

FIG. 8A is a perspective view showing the tampon completely contained by the second pocket with the finger of a user inserted through slits in the two faces of the second pocket;

FIG. 8B is a perspective view showing the second pocket being twisted;

FIG. 9A is a perspective view showing the tampon contained in the second pocket being threaded through the optional slits;

FIG. 9B is a view of a tampon fully encased in the second pocket and ready for disposal.

### DETAILED DESCRIPTION OF THE INVENTION

In the description of the invention above and in the detailed description of the invention, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Disclosed herein is a tampon enclosure system 100 for enclosing unused sanitary tampons, a user's hand during removal, and a used applicator or used sanitary tampon for disposal.

Tampon enclosure system 100 is comprised of a first pocket 110 and a second, larger pocket 120. The first pocket 110 is joined to the second pocket 120, and the pockets may be separated by a perforation 130 between the two pockets so that the first pocket 110 and the second pocket 120 can be easily separated without compromising the structural integrity of either pocket. That perforation should be adjacent to the interior seam 135 of the first pocket, and removing the first pocket from the second pocket by tearing along the perforation 130 should create the opening 145 of the second pocket 120.

First pocket and second pocket are formed by joining two sheets of material together along their peripheries. In one embodiment, the two sheets of material which make up the second pocket each form a slit 121a, 121b. Preferably, when the two sheets of material are joined to form the second pocket, the slits can be aligned.

One sheet of material of the second pocket 120 may define a protruding enclosure 122. The protruding enclosure 122 may be located approximately midline on a vertical transverse axis and below the horizontal transverse axis with respect to the second pocket 120.

The first pocket 110 is sized and shaped to accommodate a tampon 105. The second pocket 120 is sized and shaped to accommodate a user's hand. The enclosure system including both pockets may be approximately 6 inches by approximately 9 inches, and the first pocket may be approximately 1 inch by 6 inches, although those measurements are offered by way of illustration and not limitation. The protruding enclosure 122 is enclosed on its periphery and open and in communication with the second pocket, and is sized and shaped in a manner appropriate for encasing the thumb of a user's hand when the user's hand is inserted into the second pocket 120. The seams of the first pocket 110 and the second pocket 120 and the protruding enclosure may be formed through heat sealing or other means. At least one seam 140 of the first pocket 110 may be perforated or otherwise easy to open so that a tampon is easily removed from the pocket.

Preferably, the tampon enclosure system 100 is made of a thin polymer material such as polyethylene or polylactic acid. One appropriate, non-polyethylene based plastic is marketed by Navomont under the commercial name Mater-Bi. The material should be waterproof or water resistant, and it should be sufficiently thin so that it does not interfere with tactile sensation of a user's fingers so that the user can effectively remove the tampon. It can be biodegradable, but it must be able to resist abrasion caused by a prolonged period of contact with other objects in a purse or bag without permitting the tampon within it to be soiled or unduly damaged.

As shown in FIGS. 1A and 1B, a tampon enclosed in an enclosure system can further be contained in a secondary wrapper or envelope 90 which will further protect an unused tampon from damage from storage.

A method to manufacture the tampon enclosure system 100 includes the steps of: laying out a length of material, of the type or similar to the type discussed above; placing a tampon towards the center of the length of material; and folding the length of material over itself. The first pocket 110 and the second pocket 120 are formed by heat sealing or stamping edges. A perforation 130 may be added between the first and second pockets so that they are detachable from one another. The material may define the protruding member 122, which may be molded into one sheet of material, so that the protruding member 122 will lie on the second pocket 120 when the tampon enclosure system 100 is formed.

When packaged for sale, the tampon enclosure system 100 may house a tampon 105 in the first pocket 110. Once a tampon is housed in the first pocket 110, it is sealed in a manner generally known and used to seal polymers or other materials that may be used to manufacture the tampon enclosure system 100. The second pocket 120 is then wrapped around the tampon and the first pocket 110. Optionally, the wrapped tampon enclosure system 100 may be held in place using a band of paper 160, for example. Alternatively, the wrapped tampon enclosure system 100 may be packaged in a secondary wrapper 90.

Referring to FIG. 3, a user separates the first pocket 110 from the second pocket 120 along the perforation between the two pockets 130, and then removes the tampon from the second pocket when ready to use it. Referring to FIG. 5, in order to remove and dispose of a tampon, the user may insert her hand into the second pocket 120 through the opening 145 formed when the first pocket 110 is detached from the second pocket 120. The user can extend her thumb into the protruding enclosure 122, if present. Referring to FIGS. 6A through 9B, using the protected hand, the user finds the string of the tampon 105 requiring removal and removes the tampon. While still holding the string, the user pulls the second pocket 120 off of her hand and inverts it over the tampon, as shown in FIGS. 6A, 6B, and 7. Referring to FIG. 8A, the tampon is then enclosed in the inverted second pocket. It can be secured in the inverted second pocket by twisting the pocket around the tampon, as shown in FIG. 8B, or the pocket could be tied. Alternatively, a user can place her finger between the two slits 121a, 121b, as shown in FIG. 8A. She then twists the bag one or more times, as shown in FIG. 8B. The enclosed tampon can then be threaded through slits 121a, 121b, like a button through a button hole, as shown in FIG. 9A, creating a sealed enclosure. A used applicator can be disposed of in the same pocket.

The tampon 105 in the first pocket 110 may be removed and used immediately, or it may be kept in the first pocket 110 for future use. The first pocket 110 will protect the tampon from damage by articles it encounters during this period of storage.

Alternative embodiments of the tampon enclosures system disclosed and described herein may enclose a portion of a user's hand, may have squared corners instead of rounded ones, and may be manufactured in different ways. The first pocket may be omitted, and rather the new tampon may be enclosed by wrapping the second pocket around it. The protruding enclosure may be absent, and the second pocket may take an alternative shape, such as a V shape, with each of thumb and fingers inserted in the branches of the V during use. The second enclosure may have squared corners, and a user's thumb and fingers may be inserted into the squared corners during use. The slits may be omitted and alternative means of fastening the second pocket around the tampon, such as adhesive tabs, ties, or drawstrings may be employed. Variations of this nature are considered to be within the scope of the system disclosed herein.

The terms and expressions which have been used in this specification are intended to describe the invention, not limit it. The scope of the invention is defined and limited only by the following claims.

1. A tampon enclosure system comprising:
a tampon;
a first pocket and a second pocket, said first pocket being removably joined to the second pocket;
said first pocket being sized and shaped to accommodate said tampon; and
said second pocket being sized and shaped to accommodate a hand of a tampon user.

2. The tampon enclosure system of claim 1 where said first pocket is joined to said second pocket by perforated material.

3. The tampon enclosure system of claim 1 wherein said second pocket comprises two sheets of material, and each said sheet of material defines at least one slit.

4. The tampon enclosure system of claim 3 wherein one said sheet of material defines a protruding enclosure which is sized and shaped to accommodate a user's thumb.

5. The tampon enclosure system of claim 1 wherein said enclosure system is formed from polyethylene.

6. The tampon enclosure system of claim 1 wherein said enclosure system is formed from polylactic acid.

7. The tampon enclosure system of claim 1 wherein said enclosure system is biodegradable.

8. The tampon enclosure system of claim 1 wherein said enclosure system is formed from a moisture resistant material.
